(19) United States
(12) Patent Application Publication
(10) Pub. No.: US 2019/0209101 A1
(43) Pub. Date: Jul. 11, 2019

(54) APPARATUS FOR ANALYZING TELE-REHABILITATION AND METHOD THEREFOR

(71) Applicant: UINCARE Corporation, Seoul (KR)

(72) Inventors: Sung Kyoon LEE, Seongnam-si (KR); Yong Tae WON, Osan-si (KR); Dong Won KANG, Seoul (KR)

(21) Appl. No.: 15/927,930

(22) Filed: Mar. 21, 2018

(30) Foreign Application Priority Data
Jan. 10, 2018 (KR) 10-2018-0003543

(51) Int. Cl. A61B 5/00; G06T 7/00; G06K 9/00; G06K 9/62

(52) U.S. Cl. CPC A61B 5/7465; A61B 5/0013; A61B 5/0022; A61B 5/0077; G06T 2207/30196; G06T 7/0012; G06K 9/00342; G06K 9/628; A61B 2505/09; A61B 5/742

(57) ABSTRACT

Provided is a method of analyzing tele-rehabilitation which includes calculating a completion rate of the number of times of performance, indicating how much a user achieves a designated number of times of performance of a first rehabilitation exercise, based on the designated number of times of performance of the first rehabilitation exercise, and the number of times of the first rehabilitation exercise actually performed by the user, by an apparatus for analyzing tele-rehabilitation, calculating a variation coefficient indicating whether the first rehabilitation exercise is regularly and repeatedly performed, based on a performance time consumed at each time while the user repeatedly performs the first rehabilitation exercise, by the apparatus for analyzing tele-rehabilitation, and generating a result of analysis indicating a result obtained as the user performs the first rehabilitation exercise, based on the completion rate of the number of times of performance and the variation coefficient, by the apparatus for analyzing tele-rehabilitation.

### CROSS-REFERENCE TO RELATED APPLICATIONS

This application claims the priority of Korean Patent Application No. 10-2018-0003543 filed on Jan. 10, 2018, in the Korean Intellectual Property Office, the disclosure of which is incorporated herein by reference.

### BACKGROUND

### Field

The present disclosure relates to an apparatus for analyzing tele-rehabilitation and a method therefor. The present disclosure relates more particularly to an apparatus for analyzing tele-rehabilitation, which, when a user remotely performs a rehabilitation exercise, analyzes a result of the rehabilitation exercise and provides information on the analysis to the user or a rehabilitation exercise manager, and a method therefor.

### Description of the Related Art

In recent years, as we enter an aging society, the number of cerebral stroke patients has increased, the number of spinal cord injured patients resulting from traffic accidents has increased, and the number of patients requiring rehabilitation treatments due to various diseases has increased.

However, long-term inpatient treatments, nursing, and the like of rehabilitation patients increase caregiving burdens of families, and increase personal and social costs consumed in association with aftereffects and complications. In particular, a large number of chronic rehabilitation patients leave hospitals while severe dysfunction is maintained, and receive outpatient treatments.

Nevertheless, rehabilitation is very difficult for the chronic rehabilitation patients receiving the outpatient treatments due to lack of professional staffs to nurse the chronic rehabilitation patients during non-hospital hours and due to lack of knowledge of families of the chronic rehabilitation patients about nursing and rehabilitation. Tele-rehabilitation is attempted as a method of solving the limited number of times of treatments and burdens of social and economic costs of the chronic rehabilitation patients.

The tele-rehabilitation refers to remotely providing a comprehensive rehabilitation service to patients who are difficult to receive a rehabilitation service due to a distance from a medical facility and patients who want to receive treatments in home. In particular, patients who do not have guardians and patients who are difficult to go to a hospital feel difficulty in rehabilitation. The tele-rehabilitation may provide an effective rehabilitation treatment to these patients using a device and a communication network.

When the existing rehabilitation is remotely managed, in most cases, rehabilitation exercise contents (image), and the like are provided to notify of an exercise method that may be performed in home and to perform exercise counseling and prescription through an image. In this case, there is an advantage in that an exercise may be managed through constructing a relatively simple system in home of a patient, but there is a difficulty in identifying whether patients perform exercises and whether a large number of object persons are systematically managed.

In recent tele-rehabilitation, virtual reality using a three-dimensional image measuring device is implemented and the exercise guide and management using a program is performed through measuring an exercise posture to prove that the tele-rehabilitation is clinically effective for various diseases. However, in this method, expensive system construction costs are consumed when the tele-rehabilitation is performed in home, and thus a burden of rehabilitation costs may increase.

Accordingly, when the tele-rehabilitation is performed in home, a system which may easily analyze and monitor rehabilitation with a low-price system has been required more and more.

### SUMMARY

An aspect of the present disclosure is to provide an apparatus for analyzing tele-rehabilitation and a method therefor.

Problems of the present disclosure are not limited to the above-described problem(s), and other not-described problems could be clearly understood by those skilled in the art with reference to the following descriptions.

To solve the technical problem, a method of analyzing tele-rehabilitation according to an embodiment of the present disclosure may include calculating a completion rate of the number of times of performance, indicating how much a user achieves a designated number of times of performance of a first rehabilitation exercise, based on the designated number of times of performance of the first rehabilitation exercise, and the number of times of the first rehabilitation exercise actually performed by the user, by an apparatus for analyzing tele-rehabilitation, calculating a variation coefficient indicating whether the first rehabilitation exercise is regularly and repeatedly performed, based on a performance time consumed at each time while the user repeatedly performs the first rehabilitation exercise, by the apparatus for analyzing tele-rehabilitation, and generating a result of analysis indicating a result obtained as the user performs the first rehabilitation exercise, based on the completion rate of the number of times of performance and the variation coefficient, by the apparatus for analyzing tele-rehabilitation.

Preferably, the calculating of the completion rate of the number of times of performance may include calculating the completion rate of the number of times of performance as a value obtained by dividing the number of times of actual performance by the designated number of times of performance.

Preferably, the calculating of the variation coefficient may include calculating a mean and a standard deviation of the performance time consumed at each time, and calculating the variation coefficient as a value obtained by dividing the standard deviation by the mean.

Preferably, the generating of the result of the analysis may include calculating a rehabilitation exercise index as a value obtained by dividing the completion rate of the number of times of performance by the variation coefficient.

Preferably, the method may further include calculating the number of times of actual performance and the performance time consumed at each time, based on a plurality of images photographed while the user repeatedly performs the first rehabilitation exercise, by the apparatus for analyzing tele-rehabilitation.

Preferably, the calculating of the number of times of actual performance and the performance time consumed at each time may include estimating the number of images photographed at every arrival time while the first rehabilitation exercise having a start-arrival-end cycle is repeatedly performed as the number of times of actual performance, and estimating a difference between photographing times of the plurality of images as the performance time consumed at each time.

Preferably, the method may further include classifying the plurality of images into a group A corresponding to images obtained by photographing a main movement and a group B corresponding to the other images, based on a correlation coefficient indicating a similarity between the plurality of images, and correcting the result of analysis based on a main operation occupancy rate indicating a ratio of the number of the image belonging to the group A to the number of the plurality of images.

Preferably, the correcting of the result of the analysis may include calculating the rehabilitation exercise index as a value obtained by dividing the completion rate of the number of times of performance by the variation coefficient and multiplying a resultant value by the main operation occupancy rate.

Preferably, the method may further include when the rehabilitation exercise index exceeds a predetermined value, guiding a rehabilitation exercise manager such that the rehabilitation exercise manager performs a rehabilitation exercise having a higher difficulty level than that of the first rehabilitation exercise.

Preferably, the generating of the result of the analysis may include generating a first overlapping image corresponding to one image obtained as the images belonging to the group A overlap with each other, and generating a second overlapping image corresponding to one image obtained as the images belonging to the group B overlap with each other.

To solve the technical problem, an apparatus for analyzing tele-rehabilitation according to another embodiment of the present disclosure may include a display unit configured to reproduce rehabilitation exercise contents such that a user copies a first rehabilitation exercise, a measurement unit configured to measure a movement of the user who copies the rehabilitation exercise content, and an analysis unit configured to calculate a completion rate of the number of times of performance, indicating how much a user achieves a designated number of times of performance of a first rehabilitation exercise, based on the designated number of times of performance of the first rehabilitation exercise, and the number of times of the first rehabilitation exercise actually performed by the user, calculating a variation coefficient indicating whether the first rehabilitation exercise is regularly and repeatedly performed, based on a performance time consumed at each time while the user repeatedly performs the first rehabilitation exercise, and generating a result of the analysis indicating a result obtained as the user performs the first rehabilitation exercise, based on the completion rate of the number of times of performance and the variation coefficient.

Using the present disclosure, when tele-rehabilitation is performed in home, a low-price system using a device having a camera mounted thereon is constructed to analyze a rehabilitation exercise of a patient and to transmit a result of the analysis to an exercise manager.

In particular, the patient performs an exercise through rehabilitation exercise contents (image guide) mounted on a mobile computer, generation of an event is guided through a voice or a remote controller when the patient maintains a specific movement, and when a specific event occurs, image data and an image shooting time are acquired through a camera and are used to analyze the rehabilitation exercise.

Through this, a user may perform rehabilitation in home through a tele-rehabilitation analyzing apparatus constructed with low costs even when he/she doesn't visit a hospital. Also, the tele-rehabilitation analyzing apparatus may analyze a result of a rehabilitation exercise and provide a rehabilitation exercise customized with a patient, thereby effectively helping rehabilitation of the patient.

Effects of the present disclosure are not limited to the above-described effects, and other not-mentioned effects could be clearly understood by those skilled in the art with reference to the following description.

### BRIEF DESCRIPTION OF THE DRAWINGS

The above and other aspects, features and other advantages of the present disclosure will be more clearly understood from the following detailed description taken in conjunction with the accompanying drawings, in which:

FIG. 1 is a view for explaining a tele-rehabilitation analyzing apparatus according to an embodiment of the present disclosure;

FIG. 2 is a view for explaining a configuration of the tele-rehabilitation analyzing apparatus according to the embodiment of the present disclosure in more detail;

FIG. 3 is a view for explaining a method of analyzing the tele-rehabilitation according to the embodiment of the present disclosure;

FIGS. 4 and 5 are views for explaining an image data analyzing process used in the embodiment of the present disclosure;

FIG. 6 is a view for explaining an image shooting time analyzing process used in the embodiment of the present disclosure;

FIGS. 7 to 9 are views for explaining a method of analyzing tele-rehabilitation according to the embodiment of the present disclosure; and

FIG. 10 is a block diagram illustrating hardware of the tele-rehabilitation analyzing apparatus according to the embodiment of the present disclosure.

### DETAILED DESCRIPTION OF THE PREFERRED EMBODIMENT

The present disclosure may be variously changed and may have various embodiments, and thus specific embodiments will be illustrated in the drawings and will be described in detail. However, it should be understood that this is not intended to limit a specific embodiment and includes all changes, equivalents, and substitutions included in the spirit and the technical scope of the present disclosure. In describing each drawing, similar reference numerals are designated by similar components.

Although terms such as first, second, A and B may be used to describe various components, the components are not limited by the terms. The terms are used only to distinguish one component from other component(s). For example, a first component may be named a second component without departing from the scope of the right of the present disclosure.

Similarly, the second component may be named the first component. Terms such as and/or include a combination of a plurality of related described items or any item of the plurality of related described items.

It should be understood that when it is mentioned that a first component is “connected to” is or “jointed to” a second component, the first component is directly connected to or joined to the second component, but a third component may be interposed therebetween. On the other hand, it should be understood that when it is mentioned that a first component is “directly connected to” or is “directly joined to” a second component, a third component is not interposed therebetween.

Terms used herein are merely used to describe a specific embodiment, and are not intended to limit the present disclosure. A singular expression may include a plural expression unless otherwise specified. In the present application, it should be understood that the term such as “include” and “have” are intended to specify that there are features, numbers, steps, operations, elements, components, or a combination thereof disclosed in the specification, but do not preclude the presence or addition of one or more other features, numbers, steps, operations, elements, components, or combinations thereof.

Unless otherwise defined, all terms used herein including technical terms and scientific terms may have the same meanings as those generally understood by those skilled in the art to which the present disclosure pertains. The terms defined in a generally used dictionary should be interpreted to have the same meanings as those in the context of the related art, and are not interpreted as ideal or excessively formal meanings unless clearly defined in the present application.

Hereinafter, exemplary embodiments of the present disclosure will be described in detail with reference to the accompanying drawings.

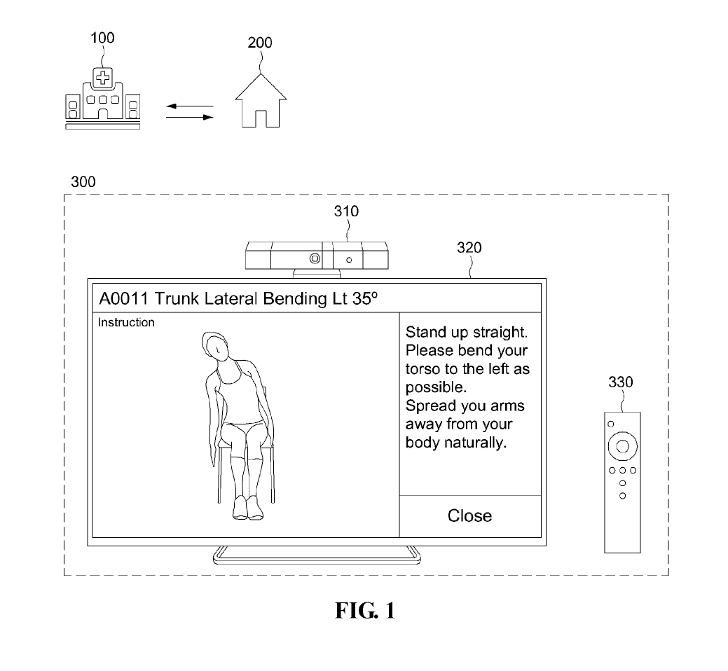

FIG. 1 is a view for explaining a tele-rehabilitation analyzing apparatus according to an embodiment of the present disclosure.

Referring to FIG. 1, a tele-rehabilitation apparatus 300 which may motivate a patient requiring a rehabilitation treatment in home 200 such that the patient may perform a rehabilitation exercise himself/herself and by which a medical staff of a hospital 100 may monitor the rehabilitation exercise may be illustrated. Here, the apparatus 300 may include a measurement unit 310, a display unit 320, an analysis unit (not illustrated), a storage unit (not illustrated), an operation unit 330, and a communication unit (not illustrated).

Here, the measurement unit 310 may be a camera. When a user repeatedly performs a rehabilitation exercise, the measurement unit 310 photographs an image of the rehabilitation exercise. The analysis unit may generate a result of the analysis using the photographed image. At this time, the result of the analysis may provide information on performance of the rehabilitation exercise and may be used as an index for scheduling the rehabilitation exercise. Descriptions thereof will be made below in more detail.

Further, the display unit 320 may be a display device such as a television (TV). Through the display unit 320, rehabilitation exercise contents may be reproduced, and the user may be guided to copy the rehabilitation exercise contents. To this end, it is preferable that the display unit 320 and the measurement unit 310 are located to be close to each other. In an example of FIG. 1, the measurement unit 310 is located at an upper end of the display unit 320. Through this, when the user copies the rehabilitation exercise contents reproduced by the display unit 320, the measurement unit 310 measures the state in which the user copies the rehabilitation exercise contents.

Further, the operation unit 330 may be a user-portable device such as a remote controller. A small device capable of wireless communication in addition to the remote controller may be used as the operation unit 330. Further, the storage unit may store the rehabilitation exercise contents or the result of the analysis. The rehabilitation exercise contents may be downloaded from a separate server through the communication unit. Also, the result of the analysis may be uploaded to an external server through the communication unit.

Further, although the tele-rehabilitation apparatus 300 is illustrated in the form of a TV in FIG. 1, in some cases, the tele-rehabilitation apparatus 300 may be configured by a smart phone. For example, a front camera of the smart phone may substitute for the measurement unit 310, a touch screen of the smart phone may substitute for the display unit 320, a separate wireless communication device such as a voice recognition module and a BLE may substitute for the operation unit 330. When the user gives a “photographing” instruction through voice, the user performing the rehabilitation exercise may be photographed. Descriptions thereof will be made below in more detail with reference to FIG. 10.

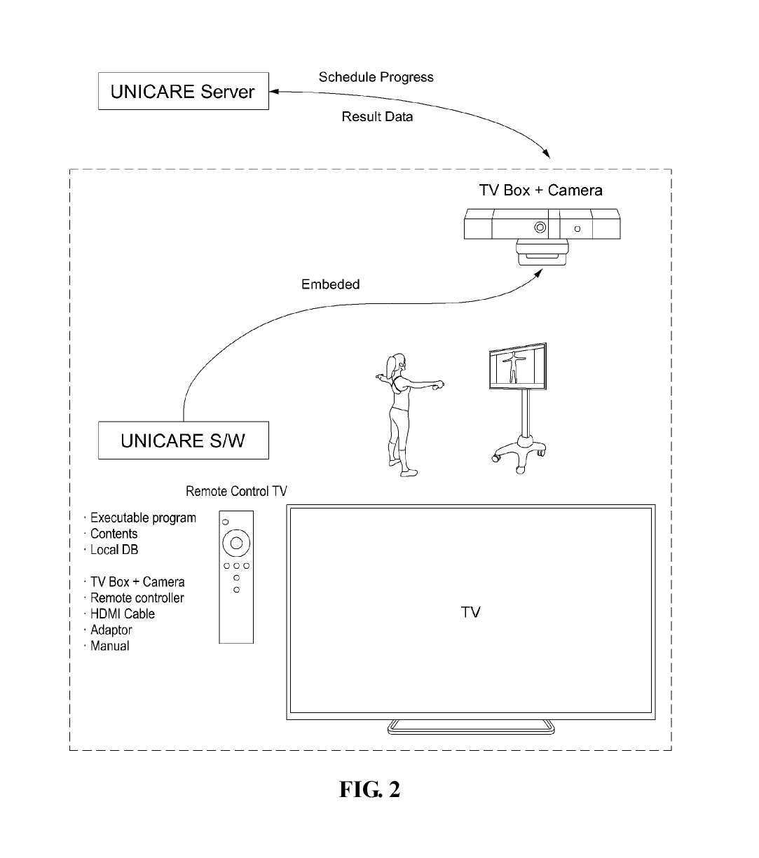

FIG. 2 is a view for explaining a configuration of the tele-rehabilitation analyzing apparatus according to the embodiment of the present disclosure in more detail.

Referring to FIG. 2, the rehabilitation exercise contents (schedule progress) are transmitted from a server 110 for providing rehabilitation exercise contents, to the tele-rehabilitation apparatus 300 of the home 200. Of course, thereafter, a result of the rehabilitation exercise performed by the user (result data) is transmitted from the tele-rehabilitation apparatus 300 to the server 110.

Here, the server 110 may be a server operated in a hospital or a server operated by a company which manages the rehabilitation exercise contents, analyzes the result of the rehabilitation exercise performed by the user, and provides the analysis to the medical staff of the hospital 100. In this case, the medical staff of the hospital 100 may access the server 110 through a personal computer or a mobile terminal to identify the analyzing result of the rehabilitation exercise performed by the user.

Next, the tele-rehabilitation apparatus 300 may provide, to the user, the rehabilitation exercise contents received from the server 110, through the display unit 320, and the user may copy the rehabilitation exercise while viewing the rehabilitation exercise contents. At this time, the user may select an image capturing button while performing the rehabilitation exercise with holding the operation unit 330 for capturing an image.

Alternatively, when the user speaks a specific voice such as “photographing” designated to photograph an image while performing the rehabilitation exercise, an image during the rehabilitation exercise may be automatically photographed through voice recognition. Such a photographed image and the rehabilitation exercise contents received from the server 110 may be stored in the storage unit (not illustrated) of the tele-rehabilitation apparatus 300.

Among them, the image obtained by photographing the rehabilitation exercise of the user may be transmitted to the server 110 in turn, and may be utilized as information for analyzing the rehabilitation exercise of the user. However, rather than this process, it is more preferable that in consideration of costs of network transmission, the tele-rehabilitation apparatus 300 analyzes the image obtained by photographing the user, and transmits only a result of the analysis to the server 110.

A process of analyzing the image obtained by photographing the user and generating the result of the analysis by the tele-rehabilitation apparatus 300 will be described in more detail with reference to the following drawings.

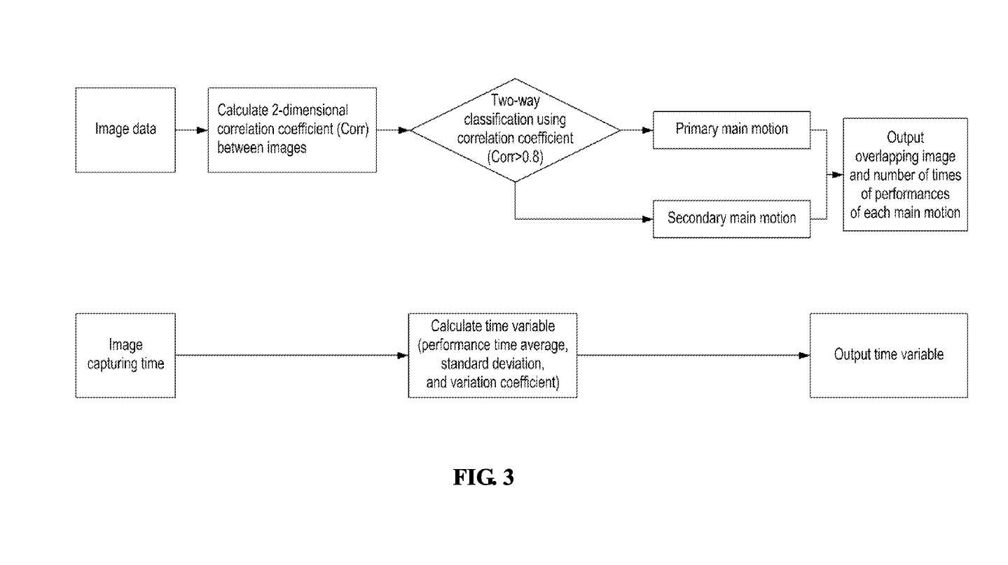

FIG. 3 is a view for explaining a method of analyzing the tele-rehabilitation according to the embodiment of the present disclosure.

Referring to FIG. 3, the tele-rehabilitation apparatus 300 may obtain two information elements from an image photographed by the camera corresponding to the measurement unit 310. One information element is photographed image data, and the other information element is a value of an image photographed time corresponding to metadata of the photographed image data. In the image data, a two-dimensional correlation coefficient between images is calculated, and the images are classified into a primary main movement and a secondary main movement with respect to a predetermined value. In an example of FIG. 3, the predetermined value is 0.8. That is, very similar images having a correlation coefficient between images of 0.8 or more are classified into the primary main movement, and images having a correlation coefficient of 0.8 or less with respect to the images belonging to the primary main movement are classified into the secondary main movement. Next, an overlapping image with respect to the primary main movement and the secondary main movement is generated, and the number of times of performance is estimated based on the number of the primary main movement and the number of the secondary main movement.

For example, it is assumed that the user requiring a rehabilitation exercise for a leg repeatedly performs an exercise of lifting up the leg while being lain according to the rehabilitation exercise contents ten times. Then, an event for photographing may occur whenever the user lifts up the leg to the maximum height. For example, the user may instruct the photographing by pushing a photographing button of the remote controller or through voice recognition such as “photographing”.

Alternatively, while consistently analyzing a movement of the user, the camera may automatically photograph an image when the movement of the user becomes a movement that is similar to the rehabilitation exercise contents. However, the rehabilitation exercise has a disadvantage in that since it is difficult to perform the rehabilitation exercise similarly to the rehabilitation exercise contents every time due to physical inconvenience of the user, it is difficult to automatically photograph an image through image analyzing.

For example, when the user repeatedly performs the exercise of lifting up the leg while being laid ten times, the user may lift up the leg up to the maximum angle for the first five to six times. However, when the number of times of repeated performance increases, the user may not often lift up the leg up to the corresponding angle and lower the leg. Therefore, it is preferable to photograph the image when a specific event, that is, manipulation of the remote controller or a voice signal, occurs rather than automatically capturing the image by analyzing the movement of the user.

Next, with regard to an image photographing time, since each image indicates performance of one time of the rehabilitation exercise, when a time interval between images is calculated, a time consumed for performing the rehabilitation exercise one time may be calculated. Statistical information such as a performance time mean, a standard deviation, and a variation coefficient is generated based on the calculated time.

Analysis information based on the generated image data and analysis information based on the image photographing time may be provided to the user. Meanwhile, the analysis information may be provided to the server 110 to be provided to a manager who manages the rehabilitation exercise, for example, the medical staff, a rehabilitation therapist, and the like, and may be provided as an indicator for scheduling the rehabilitation exercise of the user.

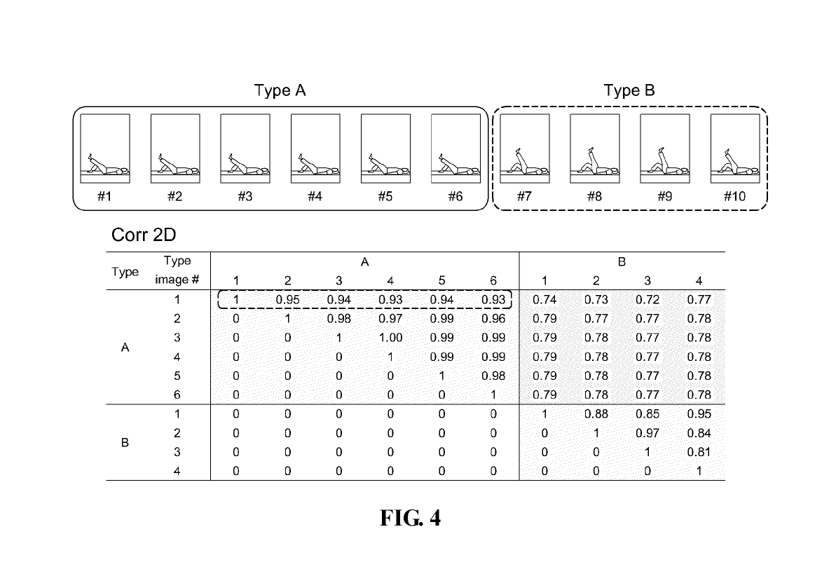

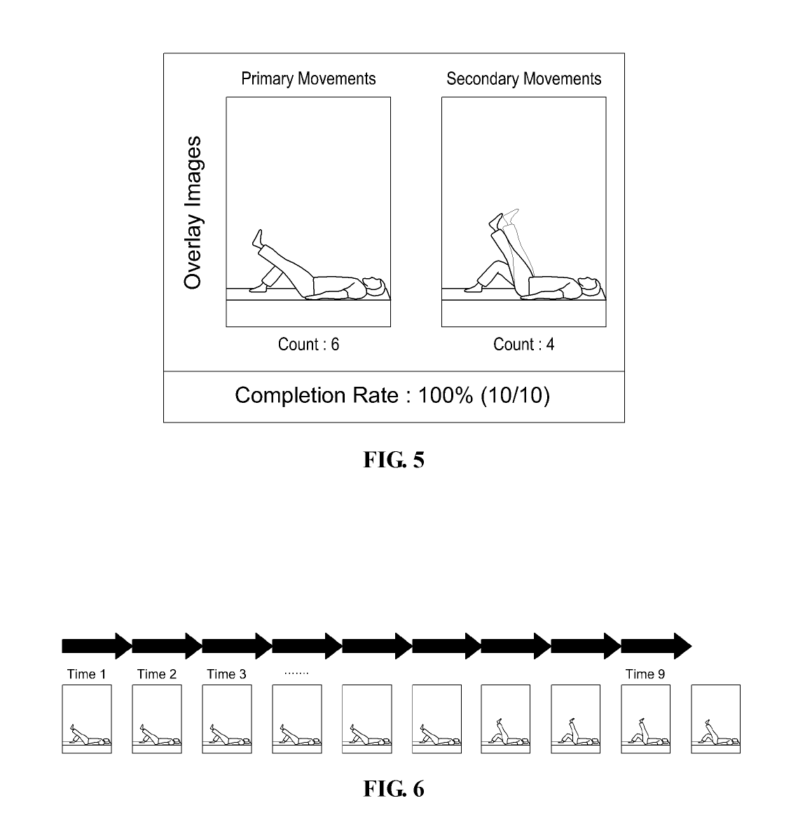

FIGS. 4 and 5 are views for explaining an image data analyzing process used in the embodiment of the present disclosure.

Referring to FIG. 4, a process of generating the analysis information based on the image data may be viewed. First, the image photographed according to the specific event is stored in the storage unit (not illustrated) in a 256-bit red-green-blue (RGB) format in matrix data. Further, the two-dimensional correlation coefficient between images is derived using the image stored in the storage unit.

At this time, an equation of deriving the two-dimensional correlation coefficient will be represented by Equation 1.

$$Corr = \frac{{\sum }_{m} {\sum }_{n} ( {A}_{mn} - \overline{A} ) ( {B}_{mn} - \overline{B} )}{\sqrt{( {\sum }_{m} {\sum }_{n} {( {A}_{mn} - \overline{A} )}^{2} ) ( {\sum }_{m} {\sum }_{n} {( {B}_{mn} - \overline{B} )}^{2} )}} \qquad [ Equation 1 ]$$

An equation of calculating a correlation coefficient between two images A and B is represented in Equation 1. Here, m and n mean a row and a column of each pixel constituting the image, and Ā and B̅ denote an entire mean of each image data. Here, for efficiency of the calculation, the correlation coefficient may be calculated using only one color among RGB colors.

For example, the correlation coefficient may be calculated using only matrix data of an R channel, the correlation coefficient may be calculated using only matrix data of a G channel, and the correlation coefficient may be calculated using only matrix data of a B channel. Through this, although the correlation coefficient is not calculated based on all colors, the correlation coefficient may be calculated using a small amount of resources.

Using the derived correlation coefficient, the image is classified into the primary main movement and the secondary main movement with respect to a reference value of 0.8. Of course, the coefficient of 0.8 used at this time may be changed to other values. That is, the correlation coefficient may be set as a proper value.

Ten images photographed using the remote controller after the user lifts up the leg to the maximum point while performing the rehabilitation exercise of lifting up the leg ten times are illustrated in FIG. 4. When a correlation coefficient between images is calculated using Equation (1), the correlation coefficient may be represented by a table displayed on a lower side of FIG. 4. That is, a correlation coefficient between a first image and a second image of a group A is 0.95, a correlation coefficient between the first image and a third image of the group A is 0.94, a correlation coefficient between the first image and a fourth image of the group A is 0.93, a correlation coefficient between the first image and a fifth image of the group A is 0.94, and a correlation coefficient between the first image and a sixth image of the group A is 0.93. In a line below a line corresponding to the first image, correlation coefficients between the second image and the third to sixth images are illustrated as 0.98, 0.97, 0.99, and 0.93. The images having the correlation coefficient between images, that is, not less than the predetermined value of 0.8 are classified into the primary main movement. These images correspond to a case where the user correctly copies the rehabilitation exercise contents, as illustrated in an upper side of FIG. 4.

In contrast, a first image to a fourth image of a group B illustrated in a lower side of FIG. 4 are images having correlation coefficients with respect to the images of the group A, which are not more than a predetermined threshold of 0.8. That is, since a correlation coefficient between the first image of the group A and the first image of the group B is 0.74, it is identified that the two images are somewhat different from each other. Likewise, a correlation coefficient between the first image of the group A and the second image of the group B is 0.73, a correlation coefficient between the first image of the group A and the third image of the group B is 0.72, and a correlation coefficient between the first image of the group A and the fourth image of the group B is 0.77.

In contrast, correlation coefficients between the images belonging to the group B exceed 0.8. That is, a correlation coefficient between the first image of the group B and the second image of the group B is 0.88, a correlation coefficient between the first image of the group B and the third image of the group B is 0.85, and a correlation coefficient between the first image of the group B and the fourth image of the group B is 0.95.

As illustrated in the upper side of FIG. 4, it can be seen that when images corresponding to a seventh rehabilitation exercise (#7), an eighth rehabilitation exercise (#8), a ninth rehabilitation exercise (#9), and a tenth rehabilitation exercise (10#), which are performed later, among the ten times of rehabilitation exercises are photographed, the images are photographed when the user fails to correctly lift up the leg to the end since the rehabilitation exercises are difficult.

In this way, when the user photographs images, correlation coefficients between images are calculated, similar images having correlation coefficients between images having a predetermined value or more are classified into groups, a group to which a larger number of images belong is classified into the primary main movement, and the other group is classified into the secondary main movement.

That is, the primary main movement means the images of the group having a high correlation coefficient between images and the highest frequency, and the secondary main movement means the other images. As illustrated in FIG. 4, the first image to the sixth image among the images photographed by the user are classified into the group A corresponding to the primary main movement, and the seventh image to the tenth image are classified into the group B corresponding to the secondary main movement.

The derived images according to the two kinds of main movements indicate overlapping images and the number of images in each group. There are advantages in that, through the overlapping images, an exercise manager may determine which movement is mainly performed while the patient performs an exercise at once through a similar image having a high frequency, and may determine the number of times of performance of the main movements through the numbers of the images included in the two groups. Also, the total number of the photographed images is used to indicate a completion rate of the number of times of performance of a customized exercise provided to the patient.

In FIG. 3, information such as the overlapping image and the number of times of performance of each main movement, which are generated based on the image data, is result data generated through the above-described process. When the result data is organized with respect to the image of FIG. 4, the overlapping images and the number of times of performance may be illustrated as in FIG. 5.

Referring to FIG. 5, the number of times of the primary main movement is 6, and when all the images corresponding to the primary main movement overlap with each other, since the images are similar to each other due to a very high correlation coefficient between images, the images are viewed as one image. As compared to this case, the number of times of the secondary main movement is 4, and when all the images corresponding to the secondary main movement overlap with each other, height angles of the leg are somewhat different from each other.

Also, ten times is obtained by adding the number of times of the primary main movement, which is 6, to the number of times of the secondary main movement, which is 4. Since the user performs all the ten times designated by the rehabilitation exercise contents of lifting up the leg, the completion rate of the number of times of performance is 100%.

Also, it can be seen that since a rate of the primary main movement is 60% obtained by dividing 6 by 10, the user correctly performs a predetermined movement only six times, and then feels difficulty in performing the movement. That is, a rate of the number of times of the primary main movement to the total number of times of performance may be utilized as an indicator indicating how much difficulty the user performs the rehabilitation exercise contents.

However, although a case where the images obtained by photographing the rehabilitation exercise are classified into two groups including the primary main movement and the secondary main movement has been described herein, the images may be classified into two or more groups according to a correlation coefficient. At this time, the images may be classified into a primary main movement to an nth main movement according to the number of images belong to the groups.

In addition, a time consumed for performing each movement may be used as an indicator indicating whether a difficulty level of the rehabilitation exercise contents is proper to the user. At this time, the consumed time is calculated based on a difference between times when images are photographed. This process will be described in more detail with reference to FIG. 6.

FIG. 6 is a view for explaining an image shooting time analyzing process used in the embodiment of the present disclosure.

Referring to FIG. 6, when totally ten images are photographed, nine values corresponding to differences between times are calculated. At this time, intervals between times when the images are photographed are performance times during which corresponding exercises are performed. Of course, in this case, a performance time of a first performed exercise may not be calculated only using the image photographing times since there is no previous time.

Instead, when a time when the first image is photographed with respect to a time when the rehabilitation exercise contents start to be reproduced is calculated, the performance time during which the first rehabilitation exercise is performed may be calculated. Alternatively, like using the voice recognition when an image is photographed, even when the rehabilitation exercise contents are reproduced, if the user speaks a voice “Start”, the contents are reproduced using the voice recognition. The performance time during which the first rehabilitation exercise is performed may be calculated using an interval between the time when the contents start to be reproduced and the time when the first image is photographed. In this way, performance times for performance units of rehabilitation exercises may be calculated, and a mean, a standard deviation, and a variation coefficient thereof may be calculated. These values may be calculated using Equation (2).

Output:

Average (mean) unit:time

Standard deviation (std) unit:time

Coefficient of Variation (Cov)=std/mean*100 unit:% [Equation 2]

In Equation (2), a mean time means a mean of performance times of exercises performed by the patient, the standard deviation means a standard deviation of the performance times of the exercise, and the variation coefficient means a variability between the performance times of the exercises.

The variation coefficient is in a range of 0% to 100%, and a low variation coefficient means that the user performs the rehabilitation exercise through a constant exercise performance time, and a high variation coefficient means an irregular exercise performance time. Thus, the exercise manager may identify a result of the exercise performance time of the patient through the following result table according to times at once.

TABLE 1

| | Mean | Std | cov |
|---|---|---|---|
| Time | 10.33 | 0.25 | 2.35 |

Referring to Table 1, it can be identified that when totally ten times of exercises are performed, a mean performance time is 10.33 seconds, a standard deviation is 0.25, and a variation coefficient is 2.35. These values mean that the user regularly performs the rehabilitation exercise, and may be provided as one indicator indicating whether the user continuously performs the rehabilitation exercise or proceeds to next rehabilitation exercise contents having a higher difficulty level since the ongoing rehabilitation exercise contents are appropriate for the corresponding user.

That is, such a result table may be represented by numerical values, graphs, and pictures, and may be provided to a screen on which the rehabilitation exercise contents are reproduced or a smart phone of the user. Also, the result table may be transmitted to the rehabilitation exercise manager through communication, and may be provided to identify a rehabilitation exercise result of the patient at once. Also, a protocol for performing a rehabilitation exercise customized with the patient through the rehabilitation exercise result is transmitted so that tele-rehabilitation may be efficiently managed.

In short, in the tele-rehabilitation analyzing apparatus according to the present disclosure, the patient performs the exercise through rehabilitation exercise contents (video guide) mounted on a mobile computer, and the like, and when the patient maintains a specific movement, an event is induced to occur through voice or the remote controller.

Next, when a specific event occurs, image data and an image photographing time are acquired through a camera and are used to analyze the rehabilitation exercise. Here, the number of times of performance of the rehabilitation exercise, the completion rate of the number of times of performance, a difficulty level of performance, and the like may be calculated through the image data. Also, the difficulty level of performance may be calculated even through the image photographing time.

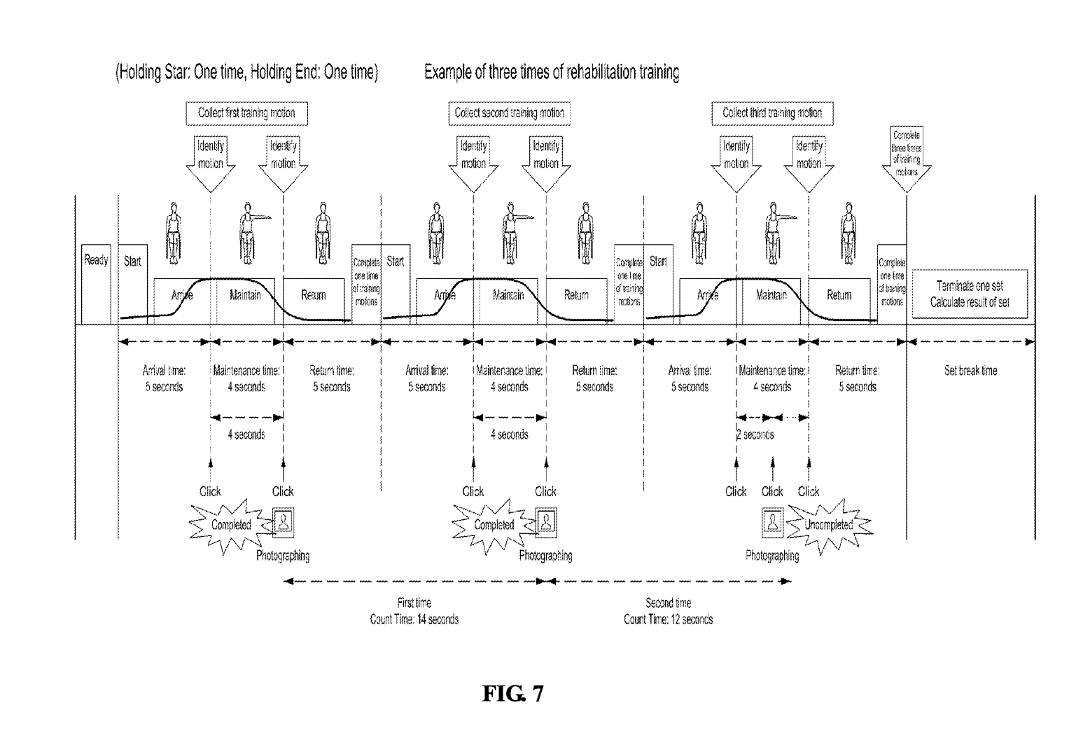

FIGS. 7 to 9 are views for explaining a method of analyzing tele-rehabilitation according to the embodiment of the present disclosure.

Referring to FIG. 7, the user performs one training movement before starting the exercise. At this time, a training movement illustrated in FIG. 7 is a movement of lifting up one arm, maintaining the arm for a specific time, and lowering the arm in turn. In this way, after the training movement of the user is recorded once, a reproduction speed of the rehabilitation exercise contents of the user may be adjusted based on the record.

For example, it is assumed that, in one time of performance, an arrival time for which the user lifts up an arm is four seconds, the user presses a start button of the remote controller while maintaining the arm lifted up, and, while maintaining the arm, photographs an image when pressing an end button at a moment when lowering the arm because it is hard to hold the arm. At this time, a maintenance time is four seconds. Finally, a lowering time for which the user lowers the arm is five seconds. Then, the rehabilitation exercise contents may be reproduced according to the reproduction speed obtained by adding five seconds, four seconds, and five seconds.

Thus, when a second training movement is performed, the reproduction speed of the rehabilitation exercise contents is adjusted, and movement information of the user is collected according to the reproduction speed. In a final third training movement, when the user photographs an image after failing to maintain the arm for four seconds and maintaining the arm for only two seconds, a performance rate at the corresponding time may be evaluated as 50% obtained by dividing two seconds by four seconds.

Since the rehabilitation exercise mostly includes a movement of maintaining an arm as well as a movement of simply lifting up and lowering the arm, this fact may be reflected to evaluate the completion rate of the number of times of performance of the rehabilitation exercise.

That is, in an example of FIG. 7, a value obtained by dividing 2.5 by 3 may be evaluated as the performance rate of an entire one set.

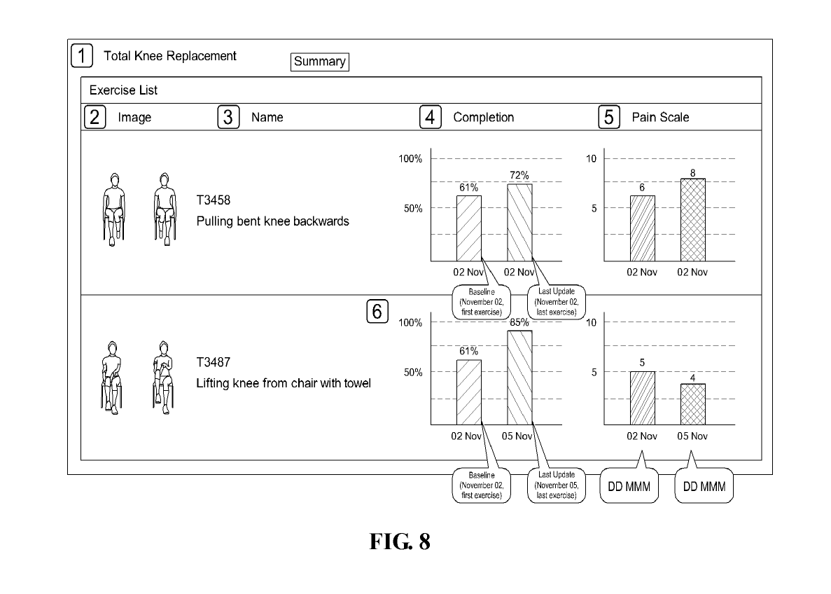

Referring to FIG. 8, an example of a user graphic screen (GUI) which provides analysis information to the user or the rehabilitation exercise manager when a knee rehabilitation exercise is performed is illustrated. A name of a rehabilitation exercise program is illustrated in number 1, and an image of rehabilitation exercise contents is illustrated in number 2.

As illustrated in an example of FIG. 8, a total knee replacement rehabilitation exercise program is a program including two kinds of movements including a movement entitled “pulling bent knee backwards” having an identifier of T3458 and a movement entitled “lifting knee from chair with towel” having an identifier of T3487. At this time, performance results of the movements are illustrated in number 4 and number 5.

Through this, a performance rate indicating how an exercise of each movement is performed by date may be provided. Also, an indicator indicating how much pain there is while the corresponding movement is performed may be provided together. Here, a pain index may be digitalized through a user survey.

Alternatively, the pain index may be automatically calculated through the image analysis based on a facial expression of the user performing the rehabilitation exercise, an interval between times generated when the movements are repeatedly performed, and a degree indicating how completely the user copies the movements while repeatedly performing the movements. For example, the pain index may be calculated based on the variation coefficient previously calculated based on the photographing time.

As illustrated in FIG. 8, when the result of the analysis is provided to the rehabilitation exercise manager, the rehabilitation exercise manager may identify how faithfully the corresponding user performs the rehabilitation exercise at once. Also, an indicator for scheduling the rehabilitation exercise based on the completion rate of the number of times of the rehabilitation exercise, the pain index, and the like, which are described above, may be provided to the manager.

For example, when the completion rate of the number of times of the rehabilitation exercise is not less than a predetermined first value, and the pain index of the user is not more than a predetermined second value, a guidance that it is preferable to proceed to a next rehabilitation exercise program may be provided. Alternatively, an index indicating a proficiency for the rehabilitation exercise of the user based on the completion rate of the number of times of the rehabilitation exercise, the pain index, and the like may be generated using Equation (3), and may be provided to the manager. Through this, the manager may provide information for scheduling the rehabilitation exercise based on the index.

$$Rehabilitation exercise index = \frac{\begin{matrix}Completion rate of \\ number of times of performance\end{matrix}}{Variation coefficient} \qquad [ Equation 3 ]$$

Here, Equation (3) may be deformed to Equation (4) including a primary main movement occupancy rate.

$$Rehabilitation exercise index = \frac{\begin{matrix}Completion rate of number of times of performance \times \\ primary main movement occupancy rate\end{matrix}}{Variation coefficient} \qquad [ Equation 4 ]$$

Also, Equation (4) may be extended to Equation (5) on which the pain index is reflected.

$$Rehabilitation exercise index = \frac{\begin{matrix}Completion rate of number of times of performance \times \\ primary main movement occupancy rate\end{matrix}}{Variation coefficient \times pain index} \qquad [ Equation 5 ]$$

Hereinafter, the description will be made based on Equation (4). In Equation (4), the completion rate of the number of times of performance is an indicator indicating that the user performs the rehabilitation exercise better as the completion rate is higher, and has a value of 0 to 100. The completion rate may be calculated through a ratio of the number of times of performance designated by the rehabilitation exercise contents and the number of times by which the user actually and repeatedly performs the rehabilitation exercise.

Next, the primary main movement occupancy rate is an indicator indicating how regularly the user repeatedly performs the rehabilitation exercise at the same posture while repeatedly performing the rehabilitation exercise, and as the primary main movement occupancy rate is higher, the user performs the rehabilitation exercise better. Likewise, the primary main movement occupancy rate has a value of 0 to 100, and may be calculated through a ratio of the number of times of the primary main movement and the total number of times of performance.

Finally, the variation coefficient is an index indicating regularity between times when the rehabilitation exercise is performed, and as the variation coefficient is lower, the user performs the rehabilitation exercise better. As described above, the variation coefficient may be calculated using a value obtained by measuring times when the rehabilitation exercise is performed at each time and dividing the standard deviation of the performance time by the mean.

Based on the rehabilitation exercise index calculated through Equation (4), when the rehabilitation exercise index is larger than a predetermined value, it meant that the user performs the rehabilitation exercise well, so that an alarm may be provided to the medical staff such that the user proceeds to a next rehabilitation exercise program, for example, a rehabilitation exercise having a higher difficulty level.

As described above, when the result of the rehabilitation exercise of lifting up the leg, which is described in FIGS. 4 and 5 as an example, is analyzed, since the completion rate of the number of times of performance has a value of 100% obtained by dividing 10 by 10, the primary main movement occupancy rate has a value of 60% obtained by dividing 6 by 10, and the variation coefficient has a value of 2.35 obtained by dividing 0.25 by 10.33, the rehabilitation exercise index has a value of 25.53% obtained by multiplying 100% by 60% and dividing a resultant value of the multiplication by 2.35.

As the user consistently and repeatedly performs the rehabilitation exercise, when the completion rate of the number of times of performance is 100%, the primary main movement occupancy rate is 90%, and the variation coefficient is 1, the rehabilitation exercise index has a value of 90% obtained by multiplying 100% by 90% and dividing a resultant value by 1. When a predetermined value is 85, the user who achieves the rehabilitation exercise of lifting up the leg at a high level may change a program to perform another program, for example, a rehabilitation exercise of lifting up not one leg but two legs, which is similar to the rehabilitation exercise of FIGS. 4 and 5.

At this time, when the rehabilitation exercise index as a determination index for changing the rehabilitation exercise program is provided to the medical staff, progress of the rehabilitation exercise performed by the user may be more simply identified as compared with a case where the progress of the rehabilitation exercise is identified through the overlapping image, the completion rate of the number of times of performance, the variation coefficient, and the like. Through this, the rehabilitation exercise index may be utilized as one of indexes for managing the rehabilitation exercise.

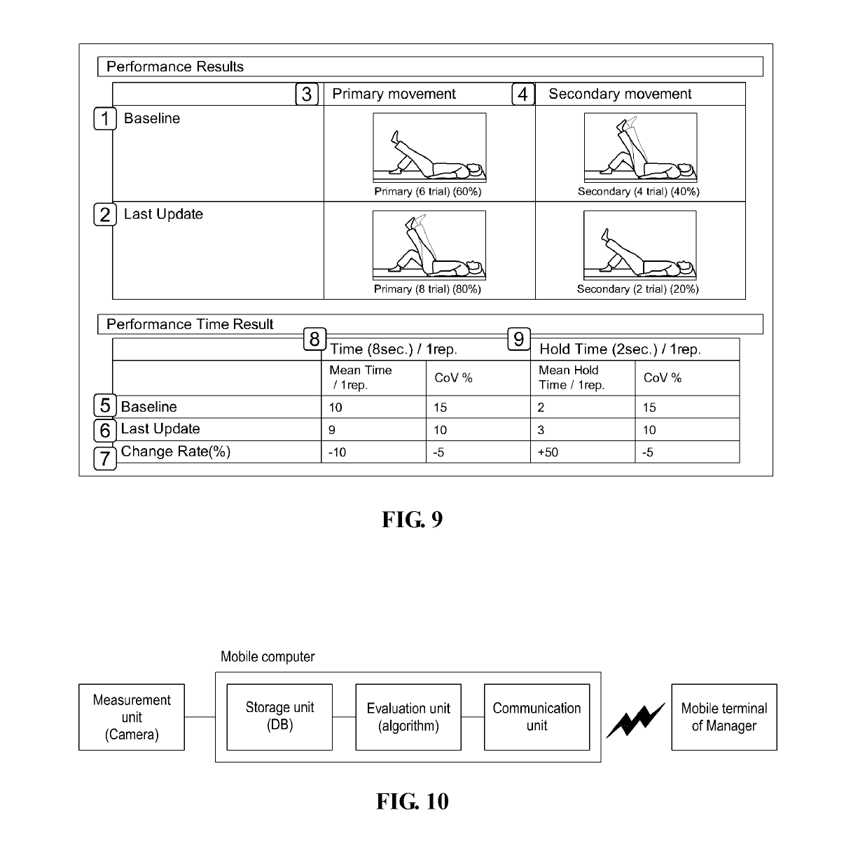

Referring to FIG. 9, the primary main movement and the secondary main movement which are described above, and the indexes such as a mean and a variance of times consumed for performing these movements may be identified. Here, statistical information on the same movement performed previously, for example, on the previous day or the day before the previous day as well as a performance result at the corresponding time are provided so that information on whether the rehabilitation exercise of the user is successful due to rehabilitation training may be provided.

In this way, the performance result of the rehabilitation exercise is managed consistently. Thus, rather than performing only one-time management, when the user simply and consistently performs the rehabilitation exercise in home, information on performance of the rehabilitation exercise may be consistently provided to the user and the medical staff to help rehabilitation of the patient.

FIG. 10 is a block diagram illustrating hardware of the tele-rehabilitation analyzing apparatus according to the embodiment of the present disclosure.

In FIGS. 1 and 2, a case where the rehabilitation exercise is performed using the TV in home has been described. However, the rehabilitation exercise may be easily implemented in home even using a mobile computer, for example, a device such as a smart phone, in addition to the TV. Here, the mobile computer refers to all devices such as a smart over the top (OTT) device, a mobile phone, a tablet PC, and a personal digital assistant (PDA), on which a mobile processor is mounted.

Referring to FIG. 10, the mobile computer may include a camera as the measurement unit, and may include the storage unit provided at an inner circumference thereof to store photographed images, rehabilitation exercise contents, and a result of the analysis.

Further, the evaluation unit may analyze images photographed by the measurement unit, generate a result of the analysis, and directly transmit the result to a mobile terminal of the manager through the communication unit. Alternatively, when the result is transmitted to the server 110, the mobile terminal of the manager may access the server 110 to identify the result.

Through such a configuration of the system, performance of the rehabilitation exercise of the patient in home may be analyzed and the manager may use the resultant information. That is, the camera in front of the patient acquires data (the image and the photographing time) according to an event (the voice or the remote controller) when a specific rehabilitation exercise is maintained, the data is analyzed, and a result for evaluating the rehabilitation exercise is derived.

Further, the image data provides an easy-to-manage monitoring result screen obtained by measuring the correlation coefficients between the images, extracting the main movements generated while the exercises are performed, and deriving the overlapping image according to the main movements so that the exercise manager may easily identify the movements generated while the patient performs the rehabilitation exercise.

Alternatively, the mean movement performance time, the standard deviation, and the variation coefficient obtained using the measured image photographing times are applied as temporal evaluation indexes for performing the exercise. Through this, the exercise manager may more easily identify a process of the rehabilitation exercise performed by the patient in a short time, and may present a customized rehabilitation exercise protocol through the result, thereby performing effective tele-rehabilitation.

Hereinabove, exemplary embodiments of the present disclosure have been mainly described. It may be understood by those skilled in the art to which the present disclosure pertains that the present disclosure may be modified without departing from the essential feature of the present disclosure. Therefore, the disclosed embodiments should be considered not in terms of limitation but in terms of description. It should be interpreted that the scope of the present disclosure is set forth not in the above description but in the appended claims, and all differences within the same scope of the appended claims are included in the present disclosure.

What is claimed is:

1. A method of analyzing tele-rehabilitation, the method comprising:
calculating a completion rate of the number of times of performance, indicating how much a user achieves a designated number of times of performance of a first rehabilitation exercise, based on the designated number of times of performance of the first rehabilitation exercise, and the number of times of the first rehabilitation exercise actually performed by the user, by an apparatus for analyzing tele-rehabilitation;
calculating a variation coefficient indicating whether the first rehabilitation exercise is regularly and repeatedly performed, based on a performance time consumed at each time while the user repeatedly performs the first rehabilitation exercise, by the apparatus for analyzing tele-rehabilitation; and
generating a result of analysis indicating a result obtained as the user performs the first rehabilitation exercise, based on the completion rate of the number of times of performance and the variation coefficient, by the apparatus for analyzing tele-rehabilitation.

2. The method of claim 1, wherein the calculating of the completion rate of the number of times of performance includes,
calculating the completion rate of the number of times of performance as a value obtained by dividing the number of times of actual performance by the designated number of times of performance.

3. The method of claim 1, wherein the calculating of the variation coefficient includes:
calculating a mean and a standard deviation of the performance time consumed at each time; and
calculating the variation coefficient as a value obtained by dividing the standard deviation by the mean.

4. The method of claim 1, wherein the generating of the result of the analysis includes,
calculating a rehabilitation exercise index as a value obtained by dividing the completion rate of the number of times of performance by the variation coefficient.

5. The method of claim 1, further comprising:
calculating the number of times of actual performance and the performance time consumed at each time, based on a plurality of images photographed while the user repeatedly performs the first rehabilitation exercise, by the apparatus for analyzing tele-rehabilitation.

6. The method of claim 5, wherein the calculating of the number of times of actual performance and the performance time consumed at each time includes:
estimating the number of images photographed at every arrival time while the first rehabilitation exercise having a start-arrival-end cycle is repeatedly performed as the number of times of actual performance; and
estimating a difference between photographing times of the plurality of images as the performance time consumed at each time.

7. The method of claim 5, further comprising:
classifying the plurality of images into a group A corresponding to images obtained by photographing a main movement and a group B corresponding to the other images, based on a correlation coefficient indicating a similarity between the plurality of images; and
correcting the result of analysis based on a main operation occupancy rate indicating a ratio of the number of the image belonging to the group A to the number of the plurality of images.

8. The method of claim 7, wherein the correcting of the result of the analysis includes,
calculating the rehabilitation exercise index as a value obtained by dividing the completion rate of the number of times of performance by the variation coefficient and multiplying a resultant value by the main operation occupancy rate.

9. The method of claim 8, further comprising:
when the rehabilitation exercise index exceeds a predetermined value, guiding a rehabilitation exercise manager such that the rehabilitation exercise manager performs a rehabilitation exercise having a higher difficulty level than that of the first rehabilitation exercise.

10. The method of claim 7, wherein the generating of the result of the analysis includes:
generating a first overlapping image corresponding to one image obtained as the images belonging to the group A overlap with each other; and
generating a second overlapping image corresponding to one image obtained as the images belonging to the group B overlap with each other.

11. An apparatus for analyzing tele-rehabilitation, the apparatus comprising:
a display unit configured to reproduce rehabilitation exercise contents such that a user copies a first rehabilitation exercise;
a measurement unit configured to measure a movement of the user who copies the rehabilitation exercise contents; and
an analysis unit configured to calculate a completion rate of the number of times of performance, indicating how much a user achieves a designated number of times of performance of a first rehabilitation exercise, based on the designated number of times of performance of the first rehabilitation exercise, and the number of times of the first rehabilitation exercise actually performed by the user, calculating a variation coefficient indicating whether the first rehabilitation exercise is regularly and repeatedly performed, based on a performance time consumed at each time while the user repeatedly performs the first rehabilitation exercise, and generating a result of analysis indicating a result obtained as the user performs the first rehabilitation exercise, based on the completion rate of the number of times of performance and the variation coefficient.
